(19) United States
(12) Patent Application Publication
(10) Pub. No.: US 2022/0211481 A1
(43) Pub. Date: Jul. 7, 2022

(54) DEVICES FOR THE TREATMENT OF PULMONARY DISORDERS WITH IMPLANTABLE VALVES

(71) Applicant: EOLO MEDICAL INC., New York, NY (US)

(72) Inventors: Sean TOTTEN, Kirkland, WA (US); Jason LEE, Milpitas, CA (US); Joe M. GUERRICABEITIA, Shoreline, WA (US); Don TANAKA, Saratoga, CA (US)

(21) Appl. No.: 17/605,978

(22) PCT Filed: Apr. 22, 2020
(86) PCT No.: PCT/US2020/029206

§ 371 Date: Oct. 22, 2021

Related U.S. Application Data
(60) Provisional application No. 62/836,762, filed on Apr. 22, 2019.

(51) Int. Cl. A61F 2/04; A61F 2/24; A61F 2/90

(52) U.S. Cl. CPC A61F 2/04; A61F 2/2418; A61F 2/2476; A61F 2250/007; A61F 2/2436; A61F 2002/046; A61F 2220/0016; A61F 2/90

(57) ABSTRACT

A flow control device for a lobar bronchial passageway including: a one-way valve; a braided wire structural frame, wherein the structural frame is expandable from a collapsed configuration to an expanded configuration; and a sealing membrane mounted to at least a distal portion of the structural frame, wherein the sealing membrane forms an enclosed wall defining at least a portion of an airflow passage through the flow control device, and the one-way valve is included in the airflow passage.

### TECHNICAL FIELD

The field of the invention is lung volume reduction devices used to treat hyper-inflated lung, for example in patients diagnosed with chronic obstructive pulmonary disease (COPD), emphysema, asthma, bronchitis. The invention relates to lung volume reduction devices such as deployable valves configured to be delivered through the airway to the lung with minimally invasive techniques.

### BACKGROUND

Hyper-inflated lung is a lung disease that makes it hard to breathe. COPD is a major cause of disability and is the third leading cause of death in the United States. The symptoms and effects of COPD often worsen over time, such as over years, and can limit the ability of a person suffering from COPD to do routine activities. Current medical techniques offer no solution for reversing the damage to the airways and lungs associated with COPD.

COPD often does not affect all air sacs or alveoli equally in a lung. A lung may have diseased regions in which the air sacs are damaged and unsuited for gas exchange. The same lung may have healthy regions (or at least relatively healthy regions) in which the air sacs continue to perform effective gas exchange. The diseased regions may be large, such as 20 to 30 percent or more of the lung volume.

The diseased regions of the lung occupy volume in the pulmonary cavity, which could otherwise be occupied by the healthy portion of the lung. If the healthy regions(s) of the lung were allowed to expand into the volume occupied by the diseased regions, the healthy regions could expand and fill with air to allow the air sacs in the healthy region to exchange oxygen for carbon dioxide.

U.S. Patent Application Publication 2014/0058433 describes methods and devices adapted for regulating fluid flow to and from a region of a patient's lung, such as to achieve a desired fluid flow dynamic to a lung region during respiration and/or to induce collapse in one or more lung regions. Pursuant to an exemplary procedure, an identified region of the lung is targeted for treatment. The targeted lung region is then bronchially isolated to regulate airflow into and/or out of the targeted lung region through one or more bronchial passageways that feed air to the targeted lung region.

U.S. Pat. No. 7,842,061 discloses an intra-bronchial device placed and anchored in an air passageway of a patient to collapse a lung portion associated with the air passageway. The device includes a support structure, an obstructing member carried by the support structure that reduces ventilation to the lung portion by preventing air from being inhaled into the lung portion, and at least one anchor carried by the support structure that anchors the obstruction device within the air passageway. The anchor may engage the air passageway wall by piercing or friction, include a stop dimensioned for limiting the piercing of the air passageway wall, and may be releasable from the air passageway for removal of the intra-bronchial device. The anchors may be carried by a peripheral portion of the support structure, or by a central portion of the support structure. The obstructing member may be a one-way valve.

W.O. International Publication Number 2004010845 discloses a flow control device for a bronchial passageway. The device can include a valve member that regulates fluid flow through the flow control device, a frame coupled to the valve member, and a membrane attached to the frame. At least a portion of the flow control device forms a seal with the interior wall of the bronchial passageway when the flow control device is implanted in the bronchial passageway. The membrane forms a fluid pathway from the seal into the valve member to direct fluid flowing through the bronchial passageway into the valve member.

However, there remains a need for a lung volume reduction device and procedure that effectively treats patients suffering from a hyperinflated lung that has improvements in affordability, implant procedure ease and speed, accessibility and removability, and safety.

### SUMMARY

This disclosure is related to methods, devices, and systems for reducing volume of a hyper-inflated lung, for example in a patient suffering from COPD.

One aspect of the disclosure is a device for reducing volume of a patient's diseased lung lobe comprising a proximal end, a distal end, a deployable structural frame, a sealing element, a valve, and a retention element. The device may be embodied as an endobronchial valve, such as a lobar one-way valve. These functions may be served by distinct structures or in some embodiments one or more structures may provide one or more of these functions.

The structural frame may further comprise a coupler on its proximal end. The coupler may be configured to mate with a delivery tool and transmit torque and translation applied to the delivery tool to the device.

The endobronchial valve, such as a one-way lobar valve, may have a sealing element that is a flexible membrane connected to the structural frame.

The endobronchial valve may include a one-way valve that permits air to flow in a direction from the distal end to the proximal end.

Also disclosed herein is a method of treating a patient with COPD comprising delivering a lobar valve through a working channel of a bronchoscope and deploying the lobar valve in a lobar bronchus that feeds a diseased lobe of the patient's lungs so that the lobar valve permits air to be released from the diseased lobe and air is not permitted to pass into the diseased lobe. The method may further comprise affixing a retention element of the lobar valve to an airway carina distal to the lobar bronchus. The retention element may be an airway carina screw or an airway carina clip. The valve may be positioned in the lobar bronchus such that the axis of the valve is not parallel with the axis of the lobar bronchus.

One or more aspects of the invention are disclosed here below:

A first aspect relates to a flow control device for a lung of a living patient, the flow control device comprising:
a wire mesh support frame, while in an expanded configuration, including:
an outer cylindrical wire mesh section with a distal circumferential edge, wherein the outer cylindrical wire mesh section is sized and configured to contact and expand a lobar bronchus in a lung of a patient;
an inner cylindrical wire mesh section with a distal circumferential edge,
an annular wire mesh section spanning and connecting proximal ends of the outer and inner cylindrical wire mesh sections,
a sealing membrane covering and/or attached to the distal circumferential edge of the outer cylindrical wire mesh section and configured to span a cross sectional area of the lobar bronchus, and
a one-way valve attached to or integrated with the sealing membrane, wherein the one-way valve includes an inlet configured to be oriented towards a downstream flow region of the lobar bronchus, wherein a flow direction is of air inhaled into the lung and flowing through the lobar bronchus, and an outlet configured to be oriented towards an upstream flow region of the lobar bronchus, and wherein the one-way valve extends into the inner cylindrical wire mesh section.

A 2nd aspect relates to the flow control device of aspect 1, wherein the sealing membrane covers at least a portion of an outer surface of the outer cylindrical wire mesh section.

A 3rd aspect relates to a flow control device for a bronchial passageway comprising:
A proximal end, a distal end, a central axis;
a wire structural frame, wherein the structural frame is transitionable between a compressed configuration and an unconstrained expanded configuration;
a sealing membrane connected to the structural frame; and
a one-way valve;

A 4th aspect relates to the flow control device of aspect 3 wherein the wire structural frame comprises at least one wire braided or woven into a generally tubular shape.

A 5th aspect relates to the flow control device of aspect 4 wherein the generally tubular shape has an ovular or elliptical cross section while in the compressed configuration.

A 6th aspect relates to the flow control device of aspect 4 wherein the wire has a diameter in a range of 0.003″ to 0.008″.

A 7th aspect relates to the flow control device of aspect 4 wherein the wire has a diameter in a range of 0.005″ to 0.006″.

An 8th aspect relates to the flow control device of any preceding aspects 4 to 7 wherein the wire structural frame comprises a braid angle in a range of 35° to 55° in the unconstrained expanded configuration.

A 9th aspect relates to the flow control device of any preceding aspects 4 to 8 wherein the wire structural frame has the generally tubular shape in the compressed configuration.

A 10th aspect relates to the flow control device of any preceding aspects 4 to 9 wherein the wire is a shape memory alloy such as Nitinol.

An 11th aspect relates to the flow control device of aspect 10 wherein the shape memory alloy has a transition temperature below 37° C.

A 12th aspect relates to the flow control device of any preceding aspects 4 to 11 wherein the wire structural frame comprises an airway wall contact region.

A 13th aspect relates to the flow control device of aspect of aspect 12 wherein the airway wall contact region is configured to conform to a surface of the bronchial passageway.

A 14th aspect relates to the flow control device of any preceding aspects 12 to 13 wherein the wall contact region is cylindrical when the flow control device is in the unconstrained expanded configuration.

A 15th aspect relates to the flow control device of any preceding aspects 12 to 13 wherein the wall contact region has a middle section with a larger diameter than proximal and distal ends of the wall contact region, when the flow control device is in the unconstrained expanded configuration.

A 16th aspect relates to the flow control device of any preceding aspects 4 to 13 wherein the at least one wire of the structural frame forms closed loop ends at the distal end of the device.

A 17th aspect relates to the flow control device of aspect 16 in combination with aspect 8 wherein the closed loop ends have a smaller angle than the braid angle.

A 18th aspect relates to the flow control device of aspect 17 wherein the smaller angle is in a range of 20° to 35°.

A 19th aspect relates to the flow control device of aspect 17 wherein the closed loop ends comprise short and long closed loop ends arranged in an alternating pattern.

A 20th aspect relates to the flow control device of aspect 19 wherein the short closed loop end has a length no greater than 2 mm and the long closed loop end has a length no less than 3.5 mm.

A 21st aspect relates to the flow control device of aspect 19 wherein the long closed loop end is in a range of 1.5 to 2 times the length of the short closed loop end.

A 22nd aspect relates to the flow control device of any preceding aspects 16 to 21, wherein the wire structural frame comprises a central axis and the closed loop ends are bent inward toward the central axis when in the expanded configuration.

A 23rd aspect relates to the flow control device of any preceding aspects 16 to 21 wherein the at least one wire comprises 48 wires braided or woven into the wire structural frame forming 24 closed loop ends on the distal end.

A 24th aspect relates to the flow control device of any preceding aspects 16 to 23 wherein the at least one wires has terminals that are gathered to form spokes at the proximal end of the support structure.

A 25th aspect relates to the flow control device of aspect 24 wherein the gathering comprises twisting, braiding, binding, holding with tubing, or gluing.

A 26th aspect relates to the flow control device of any preceding aspects 24 to 25 wherein the spokes are connected to a coupler.

A 27th aspect relates to the flow control device of any preceding aspects 3 to 25, further comprising a coupler, wherein the coupler and the spokes are a laser cut metal tube.

A 28th aspect relates to the flow control device of aspect 27, wherein the metal tube is superelastic Nitinol.

A 29th aspect relates to the flow control device of any preceding aspects 3 to 28, further comprising spokes and a coupler, wherein the spokes connect the coupler to the structural frame.

A 30th aspect relates to the flow control device of aspect 29, wherein the spokes extend from the coupler at an angle to a central axis of the wire support structure in a range of 0° to 40° in the expanded configuration.

A 31st aspect relates to the flow control device of any preceding aspects 29 to 30, wherein the spokes are curved.

A 32nd aspect relates to the flow control device of any preceding aspects 29 to 31, wherein the spokes are curved with two inflection points.

A 33rd aspect relates to the flow control device of any preceding aspects 29 to 32, wherein the spokes comprise an S-shaped curve.

A 34th aspect relates to the flow control device of any preceding aspects 29 to 33, wherein the spokes are longer than the radius of the flow control device in its unconstrained expanded configuration.

A 35th aspect relates to the flow control device of any preceding aspects 29 to 34, wherein the spokes are inverted into a lumen of the structural frame when in the unconstrained expanded state.

A 36th aspect relates to the flow control device of any preceding aspects 29 to 34, wherein the spokes are invertible into a lumen of the structural frame when in the unconstrained expanded state.

A 37th aspect relates to the flow control device of any preceding aspects 29 to 36, wherein the spokes are S-shaped in the unconstrained expanded configuration.

A 38th aspect relates to the flow control device of any preceding aspects 26 to 37, wherein the coupler is positioned at the proximal end.

A 39th aspect relates to the flow control device of any preceding aspects 26 to 38, wherein the coupler is a metal tube having a lumen.

A 40th aspect relates to the flow control device of aspect 39, wherein the spokes are crimped in the lumen of the coupler.

A 41st aspect relates to the flow control device of any preceding aspects 26 to 40, wherein the coupler comprises a threaded lumen adapted to mate with a threaded end of a delivery shaft.

A 42nd aspect relates to the flow control device of any preceding aspects 3 to 41, wherein the sealing membrane is configured to prevent or resist airflow through the bronchial passageway except through the one-way valve.

A 43rd aspect relates to the flow control device of any preceding aspects 3 to 42, wherein the sealing membrane is configured to direct a majority of bronchial passageway airflow through the one-way valve.

A 44th aspect relates to the flow control device of aspect 43, wherein the majority of bronchial passageway airflow comprises 100%, more than 90%, or more than 80% of airflow.

A 45th aspect relates to the flow control device of any preceding aspects 3 to 44, wherein the sealing membrane comprises an airway wall contact region, and a luminal covering region.

A 46th aspect relates to the flow control device of any preceding aspects 42 to 45 in combination with aspect 12, wherein the airway wall contact region of the sealing membrane is attached to at least a portion of the airway wall contact region of the structural frame.

A 47th aspect relates to the flow control device of aspect 46, wherein the sealing membrane is affixed to the wire structural frame using a bonding method comprising at least one of dip coating, laminating, spray coating, heat staking, adhesive, sewing or solvent bonding.

A 48th aspect relates to the flow control device of any preceding aspects 3 to 47, wherein the sealing membrane is made of a material comprising at least one of urethane, polyurethane, ePTFE, silicone, Parylene, or Elast-eon™.

A 49th aspect relates to the flow control device of any preceding aspects 3 to 48, wherein the sealing membrane occludes a part of an opening of the structural frame.

A 50th aspect relates to the flow control device of aspect 49, wherein a remaining part of the opening of the structural frame comprises the one-way valve.

A 51st aspect relates to the flow control device of any preceding aspects 49 to 50, wherein the opening of the structural frame is at the distal end.

A 52nd aspect relates to the flow control device of any preceding aspects 49 to 50, wherein the opening of the structural frame is at the proximal end.

A 53rd aspect relates to the flow control device of any preceding aspects 49 to 52, wherein a portion of the sealing membrane that occludes the opening has an area larger than the cross section of the opening.

A 54th aspect relates to the flow control device of any preceding aspects 3 to 53, wherein the sealing membrane is made from a material rated for up to 500% elongation.

A 55th aspect relates to the flow control device of any preceding aspects 3 to 54, wherein the sealing membrane comprises a micropatterned surface.

A 56th aspect relates to the flow control device of aspect 55 in combination with aspect 12, wherein the micropatterned surface is on an external surface of the sealing membrane at least within a portion of the membrane where the sealing membrane is connected to the wall contact area of the support structure.

A 57th aspect relates to the flow control device of any preceding aspects 55 to 56, wherein the micropatterned surface is hydrophilic.

A 58th aspect relates to the flow control device of aspect 55 wherein the micropatterned surface is on at least one of an internal surface of the sealing membrane or the one-way valve, and wherein the micropatterned surface is hydrophobic.

A 59th aspect relates to the flow control device of any preceding aspects 55 to 58, wherein the micropatterned surface comprises nanostructures molded on to the sealing membrane.

A 60th aspect relates to the flow control device of aspect 59, wherein the nanostructures comprise a plurality of pillars each having a height and width less than 1000 nanometers.

A 61st aspect relates to the flow control device of any preceding aspects 3 to 60, wherein the sealing membrane comprises a channel or opening configured to augment airflow resistance.

A 62nd aspect relates to the flow control device of aspect 61, wherein the channel is configured to allow air to flow from the proximal to distal ends and the flow control device increases resistance to air flow from the proximal to distal end.

A 63rd aspect relates to the flow control device of any preceding aspects 61 to 62, wherein the channel is configured to close over a period of a few weeks.

A 64th aspect relates to the flow control device of any preceding aspects 3 to 63, further comprising a temporary reverse flow component configured to allow air to flow from the proximal end to the distal end for a period in a range of 3 days to 3 weeks.

A 65th aspect relates to the flow control device of aspect 64, wherein the temporary reverse flow component is a biodegradable or biodissolvable component positioned in an opening of the one-way valve.

A 66th aspect relates to the flow control device of any preceding aspects 3 to 65, wherein the one-way valve is integral with the sealing membrane.

A 67th aspect relates to the flow control device of any preceding aspects 3 to 66, wherein the one-way valve and the sealing membrane are a single-piece component.

A 68th aspect relates to the flow control device of any preceding aspects 3 to 67, wherein the one-way valve is a duckbill or Heimlich valve arranged on to allow air to flow predominantly from the distal end to proximal end.

A 69th aspect relates to the flow control device of any preceding aspects 3 to 68 in combination with aspect 45, wherein the one-way valve is connected to the luminal covering region of the sealing membrane.

A 70th aspect relates to the flow control device of any preceding aspects 3 to 69, wherein the one-way valve comprises a distal flared end having a diameter in a range of 1 mm to 4 mm, or in a range of 2 mm to 3 mm.

A 71st aspect relates to the flow control device of any preceding aspects 3 to 70, wherein the one-way valve comprises a length in a range of 3 mm to 8 mm.

A 72nd aspect relates to the flow control device of any preceding aspects 3 to 71, wherein the one-way valve comprises lips that are normally open when there is no pressure differential across the on-way valve.

A 73rd aspect relates to the flow control device of any preceding aspects 3 to 72, further comprising a retention mechanism

A 74th aspect relates to the flow control device of aspect 73 wherein the retention mechanism comprises barbs extending radially from the structural frame when the flow control device is in its expanded configuration.

A 75th aspect relates to the flow control device of aspect 74, wherein the barbs protrude up to 3 mm radially from the structural frame.

A 76th aspect relates to the flow control device of any preceding aspects 74 to 75, wherein the barbs are wires having a diameter in a range of 0.003″ to 0.008″.

A 77th aspect relates to the flow control device of any preceding aspects 74 to 76, wherein the barbs are superelastic Nitinol.

A 78th aspect relates to the flow control device of any preceding aspects 74 to 77 in combination with aspect 2, wherein the barbs are a portion of the at least one wire.

A 79th aspect relates to the flow control device of any preceding aspects 74 to 78, wherein the barbs extend radially from the structural frame when the flow control device is in the compressed configuration less than when in the unconstrained expanded configuration.

An 80th aspect relates to the flow control device of any preceding aspects 74 to 75, wherein the barbs, spokes and coupler are a laser cut tube.

An 81st aspect relates to the flow control device of aspect 73, wherein the retention mechanism is a hydrophilic micropatterned surface on the sealing membrane.

An 82nd aspect relates to the flow control device of any preceding aspects 3 to 81 wherein the flow control device is configured to be delivered in a delivery sheath through a working channel of a bronchoscope.

An 83rd aspect relates to the flow control device of 82 wherein the working channel has an inner diameter in a range of 2.7 mm to 2.9 mm.

An 84th aspect relates to the flow control device of 83 having a diameter compressible to 2.6 mm or less.

An 85th aspect relates to the flow control device of any preceding aspects 3 to 84, wherein the structural frame comprises a diameter in a range of 10 mm to 18 mm in the unconstrained expanded configuration.

An 86th aspect relates to the flow control device of any preceding aspects 3 to 85 wherein the structural frame comprises a length in a range of 8 mm to 18 mm in the unconstrained expanded configuration.

An 87th aspect relates to the flow control device of any preceding aspects 3 to 86 wherein in the unconstrained expanded configuration the support structure has a diameter that is 3.8 to 7.8 times a diameter of the support structure in the compressed configuration.

An 88th aspect relates to the flow control device of any preceding aspects 3 to 87 having a length in a range of 4 to 6 mm in the unconstrained expanded configuration.

An 89th aspect relates to the flow control device of any preceding aspects 3 to 88 having a length to diameter ratio in a range of 0.5 to 0.25.

A 90th aspect relates to the flow control device of any preceding aspects 3 to 89 wherein the structural frame is made from a bioresorbable material such as a polymer matrix.

A 91st aspect relates to the flow control device of any preceding aspects 3 to 90 wherein the structural frame is expandable by a balloon.

A 92nd aspect relates to the flow control device of any preceding aspects 3 to 91 wherein the structural frame or the sealing membrane is impregnated with an agent comprising at least one of an antifungal, antibacterial, antimitotic, or anti-inflammatory agent.

A 93rd aspect relates to the flow control device of aspect 92, wherein the structural frame or sealing membrane is configured to release the agent slowly over time.

A 94th aspect relates to the flow control device of any preceding aspects 3 to 93, wherein the one-way valve is positioned at the distal end.

A 95th aspect relates to the flow control device of any preceding aspects 3 to 93, wherein the one-way valve is positioned at the proximal end.

A 96th aspect relates to the flow control device of any preceding aspects 3 to 94, wherein the structural frame comprises an inner tubular region.

A 97th aspect relates to the flow control device of aspect 96, wherein the structural frame is a shape-set tube that elastically folds to form the inner tubular region when in the unconstrained expanded configuration

A 98th aspect relates to a kit for implanting a flow control device comprising the flow control device of any preceding aspects 3 to 97, a delivery sheath, and a delivery shaft.

A 99th aspect relates to the kit of aspect 98, wherein the flow control device is provided connected to the delivery shaft.

A 100th aspect relates to the kit of any preceding aspects 98 to 99, wherein the flow control device is provided in the compressed configuration within the delivery sheath.

A 101st aspect relates to the kit of any preceding aspects 98 to 100, further comprising a loading tool.

A 102nd aspect relates to the kit of aspect 101, wherein the loading tool comprises a funnel.

A 103rd aspect relates to the kit of any preceding aspects 101 to 102, wherein the flow control device is provided in the loading tool.

A 104th aspect relates to a method of treating a patient having a hyperinflated portion of lung, comprising placing a flow control device having an unconstrained circumference in a lobar bronchus that leads to the hyperinflated portion of lung.

A 105th aspect relates to the method of aspect 104, wherein the flow control device is the flow control device of any preceding aspects 1 to 103.

A 106th aspect relates to a method to implant a flow control device in a lung of a living patient, the flow control device includes a wire mesh support frame, a sealing membrane attached to and covering a distal end of the wire mesh support frame and a one-way valve attached to the sealing membrane, the method comprising:
positioning a sheath in a lobar bronchus of the lung, wherein the flow control device is within the sheath and the wire mesh support frame is in a radially compressed configuration while in the sheath;
sliding the flow control device from a distal end of the sheath into the lobar bronchus;
expanding the wire mesh support to an unconstrained configuration in response to the flow control device being slid out of the distal end of the sheath;
positioning the sealing membrane across a cross section of the lobar bronchus as the distal end of the wire mesh support frame expands to the unconstrained configuration, and
positioning the one-way valve attached to the sealing membrane within the wire mesh support frame such that that the one-way valve has an inlet facing a downstream flow region of the lobar bronchus, wherein a flow direction is of air inhaled into the lung and flowing through the lobar bronchus, and an outlet oriented towards an upstream flow region of the lobar bronchus.

A 107th aspect relates to the method of aspect 106, wherein the wire mesh support frame is a radially compressed tube while in the sheath, and has an annular shape in the unconstrained configuration with an outer cylindrical wire mesh section, an inner cylindrical wire mesh section, an annular wire mesh section spanning the outer and inner cylindrical wire mesh sections, and in the method the positioning of the one-way valve results in the one way valve extending into the inner cylindrical wire mesh section.

A 108th aspect relates to the method of aspect 106 or 107, wherein the expansion of the wire mesh support expands the cross section of the lobar bronchus.

A 109th aspect relates to the method of aspect 108, wherein the cross section of the lobar bronchus is expended by no more than twenty percent.

A 110th aspect relates to a flow control device for a bronchial passageway comprising:
a one-way valve;
a braided wire structural frame, wherein the structural frame is expandable from a collapsed configuration to an expanded configuration; and
a sealing membrane mounted to at least a distal portion of the structural frame, wherein the sealing membrane forms an enclosed wall defining at least a portion of an airflow passage through the flow control device, and the one-way valve is included in the airflow passage.

A 111th aspect relates to the flow control device of aspect 110 further comprising barbs protruding radially outward from the structural frame while in the expanded configuration and not protruding radially outward from the structural frame while in the collapsed configuration.

A 112th aspect relates to the flow control device of aspect 111 wherein the barbs extend at an angle acute to a longitudinal axis of the flow control device.

A 113th aspect relates to the flow control device of aspect 111 or aspect 112, wherein some of the barbs are angled towards a distal end of the flow control device and others of the barbs are angled towards a proximal end of the flow control device.

A 114th aspect relates to the flow control device of any of aspects 111 to 113, wherein at least some of the barbs extend from spokes of the structural frame.

A 115th aspect relates to the flow control device of any of aspects 111 to 114, wherein at least some of the barbs extend from a middle section of the hollow structural frame.

A 116th aspect relates to the flow control device of any of aspects 111 to 114, wherein at least some of the barbs extend from a cylindrical section of the hollow structural frame, wherein the cylindrical section is at a distal portion of the flow control device.

A 117th aspect relates to the flow control device of any of aspects 110 to 116, wherein a width of the hollow structural frame in the expanded configuration is in a range of 7 mm to 12 mm or in a range of 5 mm to 15 mm or in a range of 11 mm to 14 mm.

A 118th aspect relates to the flow control device of any of aspects 110 to 117, wherein a length of the flow control device in the expanded configuration is in a range of 5 mm to 15 mm.

A 119th aspect relates to the flow control device of any of aspects 110 to 118, wherein the structural frame while in the expanded configuration, includes a cylindrical airway contact section.

A 120th aspect relates to the flow control device of aspect 119, wherein the sealing membrane is confined to the cylindrical airway contact section.

A 121st aspect relates to the flow control device of aspect 119, wherein the sealing member covers the cylindrical airway contact section and spokes included in the structural frame.

A 122nd aspect relates to the flow control device of any of aspects 110 to 121, wherein the structural frame in the collapsed configuration has a diameter no greater than 2.6 mm.

A 123rd aspect relates to the flow control device of any of aspects 110 to 122, wherein the structural frame in the collapsed configuration has a diameter in a range of 2 mm to 2.6 mm.

A 124th aspect relates to the flow control device of any of aspects 110 to 123, wherein a ratio of a length to a width of the structural frame in the expanded configuration is in a range of 0.28:1 to 0.54:1, such as about 0.417:1.

A 125th aspect relates to the flow control device of any of aspects 110 to 123, wherein a ratio of a width of the hollow structural frame in the expanded configuration to the width in the collapsed configuration is in a range of 4:1 to 7:1, such as about 5.45:1.

A 126th aspect relates to the flow control device of any of aspects 110 to 125, wherein the flow control device includes a coupler at a proximal end of the device.

A 127th aspect relates to the flow control device of any of aspects 110 to 126, wherein the flow control device includes a coupler at a proximal end of the device, and the coupler is configured to connected to a corresponding coupler of a shaft of a delivery device.

A 128th aspect relates to the flow control device of aspect 127, wherein the coupler is formed in a laser cut tube forming a proximal portion of the flow control device.

A 129th aspect relates to the flow control device of aspect 128, wherein the laser cut tube has a wall thickness in a range of 0.11 mm to 0.17 mm.

A 130th aspect relates to the flow control device of aspect 128, wherein the laser cut tube also forms spokes connected to the braided wire structural frame

A 131st aspect relates to the flow control device of any of aspects 110 to 130, wherein sealing membrane has a micropattern molded at least on the exterior surface of the airway contact section, the micropattern configured to increase water tension when contacting a wet surface.

A 132nd aspect relates to the flow control device of any of aspects 110 to 131, wherein sealing membrane has a micropattern molded on at least one of the interior surface of the airway contact section and the one-way valve, the micropattern configured to increase hydrophobic nature of the sealing membrane.

A 133rd aspect relates to the flow control device of any of aspects 110 to 132, wherein the ratio of the diameter of the flow control device in the collapsed configuration to the diameter of the flow control device in the expanded configuration is in a range of 1:10 to 2:10

A 134th aspect relates to an assembly of an air flow control device and an insertion tool for a bronchial passageway comprising:
an air flow control device, wherein each of the air flow control devices includes:
a one-way valve;
a braided wire structural frame, wherein the structural frame is expandable from a collapsed configuration to an expanded configuration;
a sealing membrane mounted to at least a distal portion of the structural frame, wherein the sealing membrane forms an enclosed wall defining at least a portion of an airflow passage through the flow control device, and the one-way valve is included in the airflow passage, and
a first coupler at a proximal end of the airflow control device;
a delivery sheath configured to be positioned in a bronchial passageway, wherein the delivery sheath includes a distal end, wherein the air flow control device, while in the collapsed configuration, is within the delivery sheath;
a delivery shaft within the delivery sheath and extends through the delivery sheath towards the distal end; and
a second coupler at the distal end of the delivery shaft, wherein the second coupler is configured to securely engage the first coupler,
wherein the delivery shaft is configured to advance through the delivery sheath to push the air flow control device from the distal end of the delivery sheath and into the bronchial passageway,
wherein the air flow control device is configured to expand from the collapsed configuration into the expanded configuration after the air flow control device is pushed out of the delivery sheath, and
wherein the air flow control device is configured to automatically release from the second coupler when an actuator on a handle of the assembly is actuated.

### BRIEF DESCRIPTION OF THE DRAWINGS

FIG. 1 is a schematic illustration of a patient's lungs and airways with the right middle lobe omitted.

FIG. 2A is a schematic illustration of a lobar valve in an unconstrained expanded state.

FIG. 2B is a schematic illustration of a lobar valve in a constrained delivery state.

FIG. 2C is a schematic illustration of a lobar valve implanted in a right upper lobar bronchus.

FIGS. 3A and 3B are schematic illustrations of closed loop ends of a braided structural frame.

FIG. 4 is a schematic illustration of a lobar valve coupler.

FIGS. 5A and 5B are schematic illustrations of a lobar valve having inverted spokes.

FIGS. 6A and 6B are schematic illustrations of a lobar valve with spokes made from separate wire loops.

FIG. 7A is a schematic illustration of a lobar valve in a constrained delivery state having undeployed retention barbs.

FIG. 7B is a schematic illustration of a lobar valve in an unconstrained state having deployed radially protruding retention barbs.

FIG. 8 is a schematic illustration of a lobar valve having a braided tubular structural frame forming an airway contact region and a valve housing.

FIG. 9A is a schematic illustration of a lobar valve having a structural frame with both ends open.

FIG. 9B is a schematic illustration of a structural frame with both ends open wherein the ends are bent inward.

FIG. 10 is a schematic illustration of a lobar valve having spokes on both proximal and distal ends.

FIG. 11 is a schematic illustration of a lobar valve formed from a tubular braided structural frame folded inward on itself.

FIG. 12 is a schematic illustration of a delivery tool holding a lobar valve in a bronchoscope.

### DETAILED DESCRIPTION

The disclosure herein is related to systems, devices, and methods for modifying air flow to and from a targeted portion of a patient's lung, which may be substantially diseased, with an implantable device in order to reduce the volume of trapped air in the targeted portion of lung, thereby increasing the elastic recoil of the remaining lung volume.

The authors conceived of and disclose herein, implantable lung volume reducing devices and medical techniques for implanting lung volume reduction devices through the trachea and bronchi, using minimally invasive deployment, bronchoscopic and surgical techniques. The device may be embodied as an endobronchial valve, such as a one-way lobar valve.

Also disclosed is a novel treatment for patients suffering from hyper-inflated lung (e.g., emphysema, COPD, bronchitis, asthma) comprising the application of a minimally invasive bronchoscopy technique to implant a lung volume reduction device into a lung airway of a patient. The implantable lung volume reduction devices, which may be generally referred to as “lobar valves” disclosed herein are intended to be placed in an airway trunk of a lobe such that a single valve regulates air flow to or from the complete lobe, which may have benefits over previously attempted valves that were intended for multiple valve placement in higher generation airways. Benefits of a lobar valve may include lower cost, faster procedure, easier implantation, easier removal, and stronger retention. However, some features of devices disclosed herein may be novel and useful for use in higher generation airways and are not limited to devices configured for placement in a trunk of a lobe.

Anatomy and Design Inputs and Challenges:

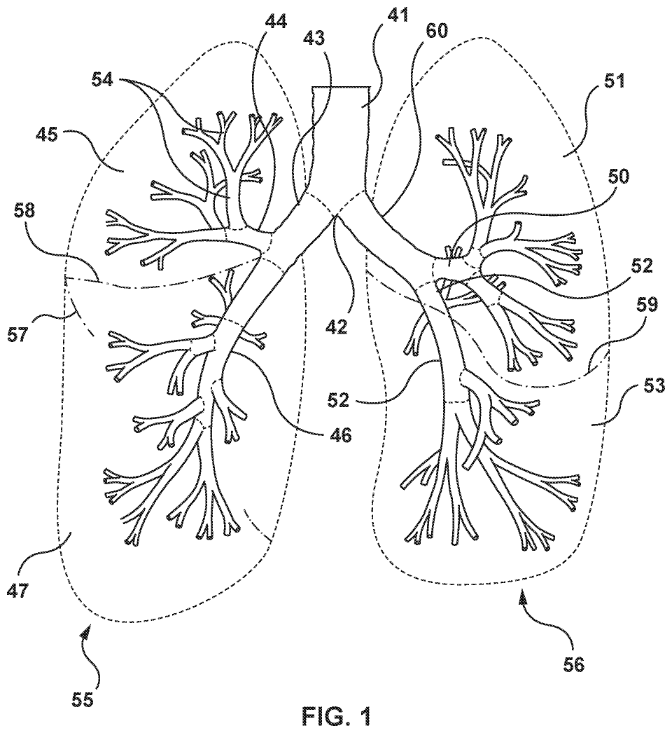

FIG. 1 is a schematic illustration of some anatomical features of human lungs. Air passes through the trachea 41, which divides into the right and left main or primary bronchi 43 and 60 between which is a carina 42. The lungs normally have clear anatomical divisions known as lobes. The right lung 55 is divided into three lobes called superior 45, middle (not shown for simplicity) and inferior 47 lobes, by the oblique 57 and horizontal 58 fissures that are folds of the visceral pleura. The left lung 56, which is slightly smaller, is divided into a superior 51 and inferior 53 lobe, by an oblique fissure 59. The term “proximal direction” refers to the direction along an airway path that points toward the patient's mouth or nose and away from the patient's lungs. In other words, the proximal direction is generally the same as the expiration direction when the patient breathes. The term “proximal section” or “proximal end” of a device implanted in a patient's airway refers to the section or end of the device intended to face the proximal direction. The term “distal direction” refers to the direction along an airway path that points toward the patient's lung or further into the lung and away from the mouth or nose. The distal direction is generally the same as the inhalation or inspiratory direction when the patient breathes. The term “distal section” or “distal end” of a device implanted in a patient's airway refers to the section or end of the device intended to face the distal direction.

Lobar valves may be implanted in a secondary bronchus, also known as a lobar bronchus. Humans have one lobar bronchus providing air passage to each lobe of the lung, including three in the right lung and two in the left lung. The right-side lobar bronchi include the right upper lobar bronchus 44, right middle lobar bronchus (not shown for simplicity), and right lower lobar bronchus 46. The left side lobar bronchi include the left upper lobar bronchus 50 and left lower lobar bronchus 52. Overlapping cartilage plates of the lobar bronchi provide structural strength to maintain patency of these bronchi. Humans may typically have lobar bronchi having an average circumference in a range of 19 mm to 56 mm. The average length is about 19 mm (e.g., in a range of about 3 to 41 mm).

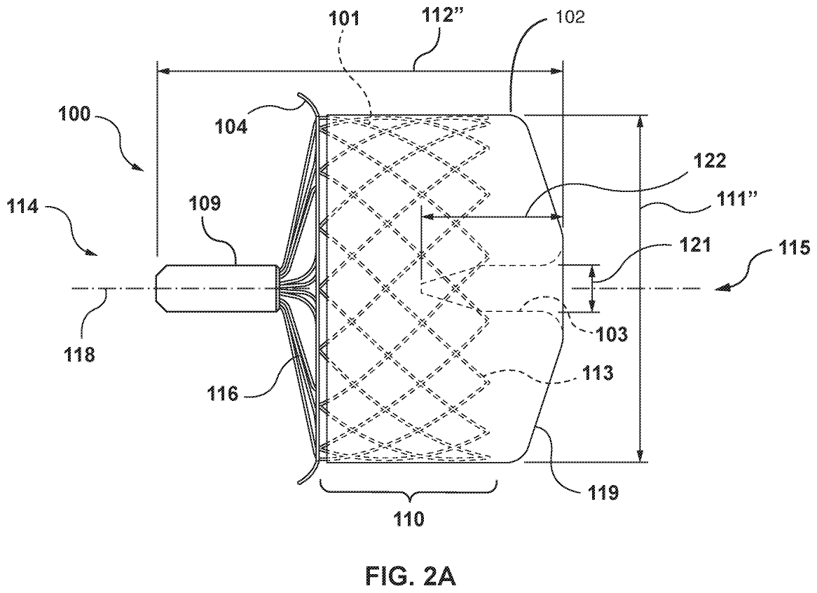

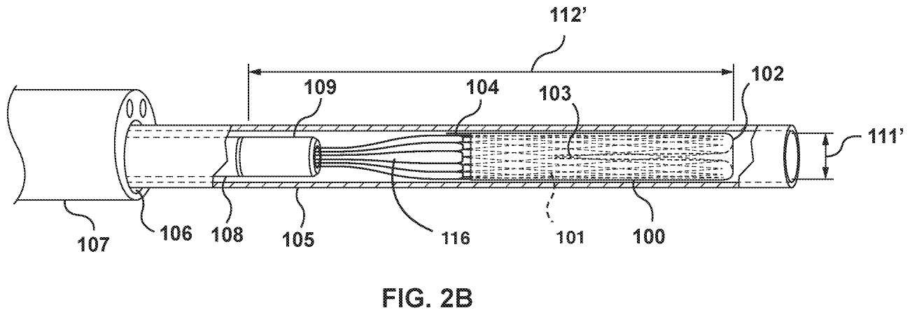

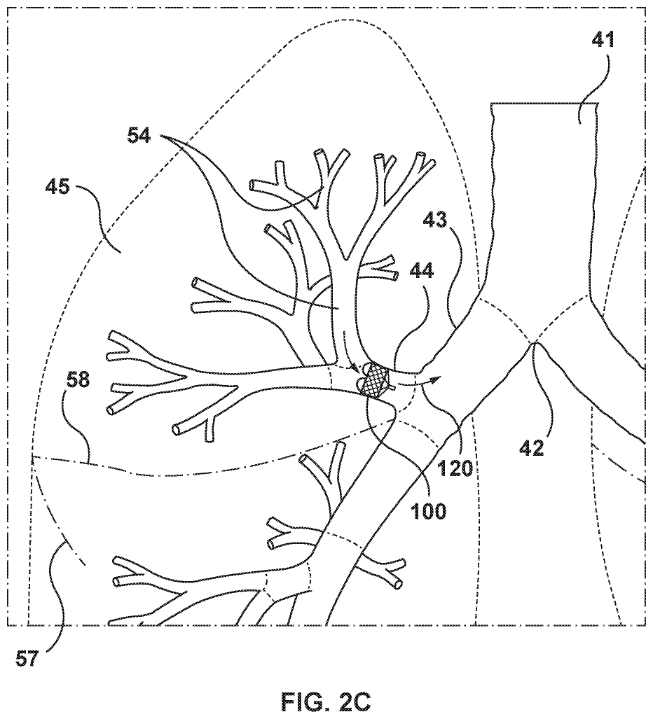

Lobar valves disclosed herein are transitionable from a contracted delivery state to an expanded deployed state. In the contracted delivery state the lobar valve is compressed and constrained in a delivery sheath that can be advanced through a bronchoscope working channel. When advanced out of the delivery sheath the lobar valve transitions to its expanded state, for example via elastic properties of a structural frame. The circumference of the lobar valve in its expanded state may be larger than the circumference of the targeted airway where it is implanted, so that a radial force is applied by the lobar valve to the airway wall. FIG. 2A is a schematic illustration showing general features of a lobar valve 100 in an unconstrained, expanded deployed state having a proximal end 114 and a distal end 115, where the distal end is intended to be implanted deeper into the lung than the proximal end. The lobar valve 100 generally comprises a structural frame 101, a sealing membrane 102, a one-way valve 103, and a retention element 104. The sealing membrane may be connected to the structural frame to function at least in part as an airway seal or an air flow control valve or an anchoring feature. The one-way valve may be part of the sealing membrane or a separate component and functions to allow fluid (e.g., air) to flow at least predominantly out of the targeted lung lobe and restrict flow into the lobe. The retention element may comprise radially extending barbs or other elements that function to hold the device in the targeted airway when exposed to forces such as lung movement and air pressure changes (e.g., coughing, sneezing, breathing).

FIG. 2B shows general features of the lobar valve 100 in a contracted delivery state where it is contained in a delivery sheath 105 that is advanced out of a distal end of a working channel 106 of a bronchoscope 107. A distal end of a delivery tool 108 is temporarily attached to a coupler 109 of the lobar valve. Various embodiments of these features may be mixed and matched and lobar valve embodiments are not limited to the combination of these elements presented in the figures.

A lobar valve may assume its contracted delivery state when delivered through a working channel of a bronchoscope, optionally contained in a delivery sheath and manipulated with a delivery tool. The lobar valve and optional delivery sheath and delivery tool may be sized to pass freely through a working channel of a bronchoscope. For example, a lobar valve adapted to be delivered with a delivery tool through a working channel with a 2.8 mm lumen may have a maximum diameter of 2.6 mm (e.g., a maximum diameter of 2.5, 2.4, 2.3, 2.2. 2.1 mm). In some embodiments lobar valves may comprise a structural frame having a delivery state and deployed state, wherein the delivery state has a maximum diameter in a range of 2 (0.0787″) to 2.5 mm (0.0984″), preferably 2.11 mm (0.083″).

Ease of use and procedural expediency is a desired requirement. The lobar valve may be designed to be consistently delivered to a correct location with average physician skill. Compared to valves that are implanted at higher generation airways implanting a lobar valve may be a faster procedure because only one valve needs to be implanted to affect an entire lobe, the lobar bronchi are larger, more proximal and hence easier to access and find than distal higher generation bronchi. Also, assessing the function of a single implanted lobar valve is faster and easier compared to assessing multiple distally implanted valves.

A lobar valve and procedure for implanting one may cost less compared to implanting multiple higher generation valves in particular since there is only one device to implant and the procedure is faster.

Design considerations may also consider particular challenges for placement in a lobar bronchus. For example, the length of a lobar bronchi is relatively short, the length to diameter ratio is considerably smaller, the cross section of a lobar bronchus is radially asymmetrical (e.g., ovular or irregular), and the diameter of the lumen is inconsistent along the length of the lobar bronchus (e.g., flared at the proximal, distal or both ends). Potentially, a single lobar valve placed in a lobar bronchus may experience a greater air pressure difference between its proximal and distal sides compared to a plurality of valves positioned in several higher generation bronchi of a lobe. Furthermore, each particular lobar bronchus in a patient has unique characteristics such as the angle of approach and geometry.

Structural Frames:

The structural frame provides a framework to hold the membrane and valve in a desired orientation and position in a target bronchus. The structural frame applies an outward radial force to press the membrane against the airway wall and hold the one-way valve in the airway so air is directed through the one-way valve.

The structural frame 101 may be made by braiding wires into at least a generally cylindrical shape. The generally cylindrical shape of the structural frame can constitute an airway wall contact region 110 that is intended to expand to contact the airway wall and to flexibly conform to the surface of the airway wall. The wires may be elastically or superelastically flexible with shape memory ability, for example the wires may be made from Nitinol that is superelastic above a temperature of body temperature (about 37° C.) or lower. As the braided wire structural frame transitions from the compressed delivery state to deployed state the device diameter (excluding optional radially extending barbs) increases from a first device diameter 111′ (FIG. 2B) toward an unconstrained second diameter 111″ (FIG. 2A); and the device length decreases from a first device length 112′ toward a second device length 112″. For example, the first device diameter 111′ may be in a range of 2 mm to 2.6 mm and the second device diameter 111″ may be in a range of 10 mm to 18 mm; and the second device length 112″ may be in a range of 8 mm to 18 mm.

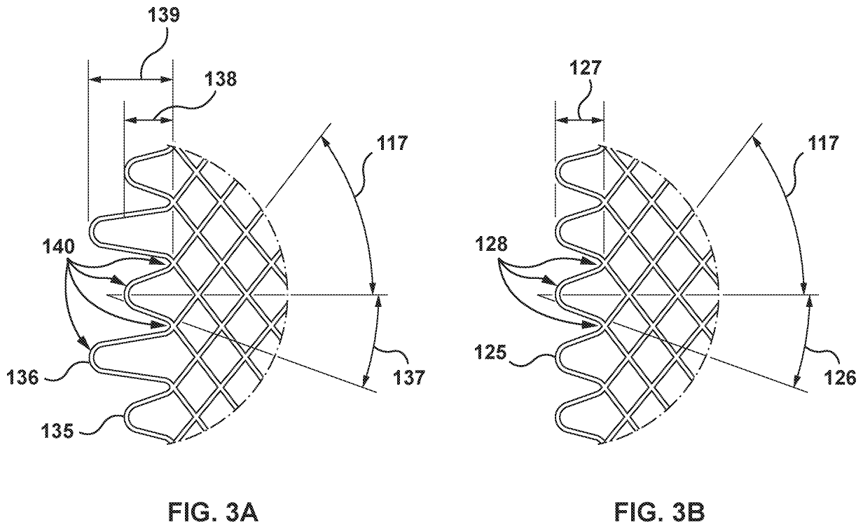

The wires used to form the structural frame braid may be for example superelastic Nitinol wires having wire diameter in a range of 0.003″ to 0.008″ (preferably in a range of 0.005″ to 0.006″). The structural frame 101 may have a braid angle 117 in a range of 35° to 55° in its unconstrained expanded configuration (see FIG. 3A). Various embodiments of braid configurations may be used without diverging from the intent of the disclosure.

In some embodiments the wires are braided with a closed loop 113 at the distal end 115 of the device as shown in FIG. 2A. For example, the structural frame 101 may have 48 wires braided with 24 closed loop ends 113 on the distal end 115 of the device and the wire terminals may gathered (e.g., twisted, braided, bound, held by tubing, glued) and shape set into spokes 116 toward the proximal end 114 and fixed into a coupler 109. Optionally, the closed loop ends may be adapted to facilitate collapse of the device 100 into its contracted delivery state. For example, as shown in FIG. 3B the closed loop ends 125 may have a smaller angle 126 (e.g., 22°, in a range of 20° to 35°) than the braid angle 117, which may require less force to bend away from its shape set configuration toward a collapsed configuration. To create a smaller angle 126 the length 127 may extended (e.g. about 2 mm) and have three points of inflection 128 (e.g., having a radius of curvature of about 0.25 mm). In another example as shown in FIG. 3A the closed loop ends may include two or more alternating closed loop end shapes such as a first closed loop end 135 and a second closed loop end 136, which may further facilitate collapse of the device by allowing the first and second closed loop ends to disperse material as they overlap in the collapsed configuration. For example, the first closed loop end 135 may be shorter than the second closed loop end 136 (e.g., the first 135 may have a length 138 of about 2 mm and the second may have a length 139 of about 3.5 mm, long closed loop end is in a range of 1.5 to 2 times the length of the short closed loop end). Both the first and second closed loop ends 135, 136 may have a reduced angle 137 as compared to the braid angle 117. To create the reduced angles 137 the wires may have three points of inflection 140 (e.g., having a radius of curvature of about 0.25 mm).

Optionally, in the unconstrained state the closed loop ends 113 may be bent inward toward the central axis to relieve forces and friction applied by the ends to the airway wall to reduce the risk of irritating the tissue which may cause granulation tissue or injury.

To accommodate lobar bronchi having an average circumference in a range of 22 mm to 44 mm multiple lobar valves may be provided. For example, a large size lobar valve may have a frame with an airway contact section having a diameter in a range of about 15 to 17 mm, preferably about 16 mm (a circumference of 50.24 mm), which may be intended to be placed in lobar bronchi having a circumference in a range of 31 mm to 44 m; and a smaller sized lobar valve may have a frame with an airway contact section having a diameter in a range of about 11 mm to 13 mm, preferably about 12 mm (a circumference of 37.7 mm), which may be intended to be placed in lobar bronchi having a circumference in a range of 22 mm to 33 mm. Note that the lobar valves may generally have a maximum unconstrained circumference that is larger than the circumference of the intended lobar bronchus (e.g., about 20 to 2.5 mm larger) so that when constrained by the lobar bronchus the airway contact section of the frame firmly contacts the airway wall and applies an outward radial force against the airway wall via the elastic properties of the structural frame and optionally other features described herein that contribute to radial contact force. The target airway may be measured using CT or other medical imaging or with a sizing device delivered through a bronchoscope.

The ratio of the maximum outer diameter of the airway contact section in an unconstrained expanded configuration to the maximum diameter of the constrained delivery configuration may be in a range of 3.8:1 to 7.8:1. Due to the relatively larger diameter and short length of lobar bronchi compared to higher generation airways, lobar valves may have a smaller length to diameter ratio in an expanded unconstrained state than current devices intended for more distal positioning. For example, a lobar valve may have a length in a range of 4 mm to 6 mm in its unconstrained state and a length to diameter ratio in a range of 0.545 to 0.286 (e.g., in a range of 0.5 to 0.25).

The structural frame along with the connected sealing membrane(s) in the delivery state may have a maximum diameter less than 2.7 mm (e.g., less than 2.6, 2.5, 2.4, 2.3, 2.2, 2.1 mm), preferably a maximum diameter of about 2.3 mm. Alternative embodiments of lobar valves may have different dimensions to allow them to be delivered through bronchoscope working channels having different diameters. Optionally, lobar valves in an unconstrained state may have a noncircular cross-section (e.g., ovoid, oval, irregular), which may have an improved fit in a bronchus having a noncircular cross-section. Alternatively, a lobar valve may be adapted to conform to a noncircular airway cross-section or irregular airway wall surface.

In situ, the structural frame may expand and contract with movement of the bronchus (e.g., during elastic recoil). The shape of the structural frame or use of its retention element may be resistant to tilting or may function properly when positioned in a range of angles with respect to the axis of the bronchus. Also, the structural frame may be compressed after it has been fully deployed allowing for repositioning. For example, a structural frame may be compressed by grasping or coupling a delivery tool to the frame's coupler and at least partially withdrawing it into a delivery sheath.

In its contracted delivery state, for example as shown in FIG. 2B, a structural frame 101 including its optional spokes 116 and coupler 109 along with a delivery sheath 105 may be sufficiently flexible to pass through a lumen 106 of an endoscope 107 (e.g., bronchoscope) when the endoscope is bent to traverse a tortuous airway (e.g., having a radius of curvature as small as 15 mm).

Optionally or alternatively, a structural frame may be made from a bioresorbable material such as a polymer matrix (e.g., PLA, PLAGA, PDLLA).

Optionally or alternatively, a structural frame may be balloon expandable or made from a plastically deformable material such as plastic, cobalt chrome alloy, martensitic Nitinol, stainless steel, silicone or urethane.

Optionally or alternatively, a structural frame may be impregnated with an agent such as an antifungal, antibacterial, antimitotic, or anti-inflammatory agent that may improve patient response to implanting the device.

In these embodiments, the wall contact region 110 may be adapted to comply to lobar bronchi that have oval or irregular lumen cross sections; the device may comply to irregular airway surfaces creating a seal on surfaces having bumps, ridges, grooves or other non-smooth surface; the device may have an overall length that is suited for fitting in lobar bronchi.

The wall contact area 110 may have flexibility and elasticity to conform to non-cylindrical (e.g., irregular, oval, tapered, flared) or non-smooth (e.g., bumpy, ridged, contoured) airways or alternatively apply a greater contact force that causes the airway wall to deform or a combination of both in order to provide a continuous circumferential sealing band to prevent air leakage in to a targeted portion of the lung under pressure differentials normally experienced in the lung. When implanted in a target airway, a structural frame may be adapted to impart an outward contact force that may expand the airway wall no more than 20% which is expected to provide strong contact and a good air seal while avoiding trauma to the tissue that otherwise could cause excess formation of granulation tissue.

Optionally, a wall contact area 110 in its unconstrained state may be barrel shaped (e.g., have a wider middle than proximal and distal ends) or be flared (e.g., have a larger diameter distally than proximally), which may facilitate creating a good contact region and seal with the airway wall.

The wall contact region 110 of the structural frame 101 provides a scaffold for the membrane 102, which is affixed to the frame, for example by dip coating, adhesive, solvent bonding or other form of bonding. The structural frame may be collapsible to its contracted delivery state in an orderly fashion that does not damage the membrane.

Spokes

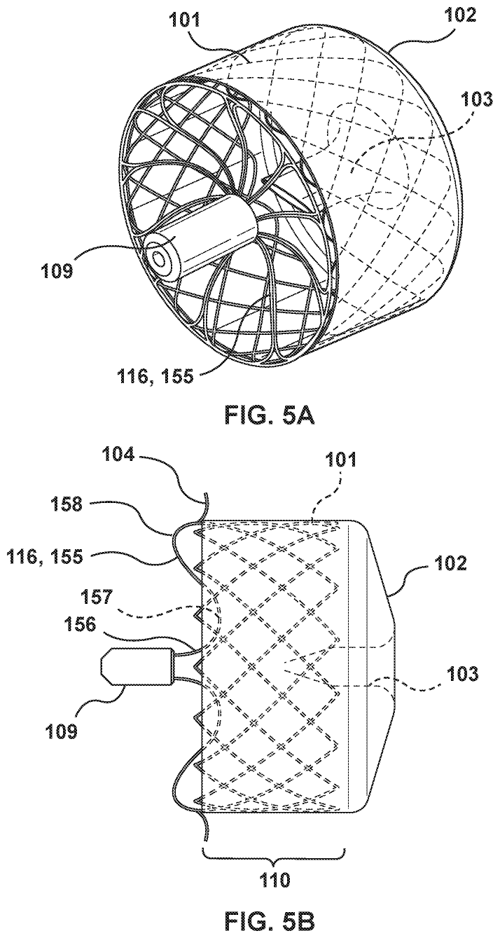

Optionally, a lobar valve may have radial spokes 116 that connect to the airway contact region 110 of the structural frame and extend inward toward the axis 118 where they may be connected to a hub or a coupler 109. In its compressed delivery state (FIG. 2B) the spokes 116 may transfer force (e.g., axially directed push or pull translation or rotation) applied to the coupler 109, for example by a delivery tool 108 attached to the coupler, to the airway contact region 110. The spokes may impart an elastic force radially outward to the airway contact region but shall not apply sufficient force to interrupt the air sealing function of the airway contact region. When a device 100 is in its expanded state and a delivery sheath 105 is advanced over the coupler 109 the force applied by the delivery sheath to the spokes 116 may cause the spokes to radially contract and collapse the airway contact region 110 allowing the device to be pulled back into the delivery sheath or to at least partially reduce the diameter of the airway contact region. This may be used to remove contact force with the airway wall to facilitate repositioning of the device. Optionally, spokes 116, 155 may have a proximal take-off section 156 (FIG. 5B) that is shape set with a concave curve or lesser angle (e.g., an angle in a range of 0° to 40° to the central axis) to the coupler than the rest of the spokes which may facilitate collapse of the device by advancing a delivery sheath which will first apply force to the take-off section to begin collapsing the spokes. In some embodiments, as shown in FIGS. 5A and 5B spokes 155 may have an “S” shaped curve that positions the coupler 109 longitudinally closer to the wall contact region 110 thereby reducing to overall length 112″ of the device when it is in its expanded state. The “S” shaped spokes 155 have a first inflection 157 and a second inflection 158. The “S” shaped curve of the spokes can impart a greater radial force between the device and airway wall, which improves retention of the device in a desired position. While implanting a device having “S” shaped spokes the device may first expand into contact with the airway wall when the sheath 105 is retracted allowing the structural frame to elastically transform to shape set configuration. Then a slight push of the delivery tool 108 may move the coupler 109 distally while the airway contact region 110 remains in place due to radial force and optionally other retention features. This can cause the “S” shaped spokes to impart an increased radial force then a decreased radial force as the coupler moves longitudinally along the axis 118 to its resting position facilitating retention and creating a haptic snap fit that can confirm the device is implanted firmly and with a proper fit.

Coupler

The proximal end of the structural frame may comprise a coupler that mates with a delivery device that allows the coupler to transmit rotational and translational force from the delivery tool to the structural frame. The coupler may be used as a graspable protrusion to grasp with a bronchoscopic tool to manipulate the device during implantation, repositioning, or removal.

For example, a lobar valve 100 may optionally have a coupler 109, positioned at the proximal end 114 of the device, that functions to mate with a coupler of a delivery shaft 108 and release from the coupler of the delivery shaft upon actuation by a user. For example, the coupler may have a geometry (e.g., male or female threading) that mates with a coupler of the delivery shaft 108. An actuator (e.g., rotary dial, trigger, slider, button) controllable by a user for example on a handle connected to the delivery sheath and delivery shaft may control the delivery shaft and sheath to control release of the couplers (e.g., retract the sheath 105 and rotate the delivery shaft 108 to unscrew the mating coupler). When attached the coupler transmits motion of the delivery shaft to the implantable valve 100 including longitudinal translation distally, proximally and rotation around longitudinal axis 118.

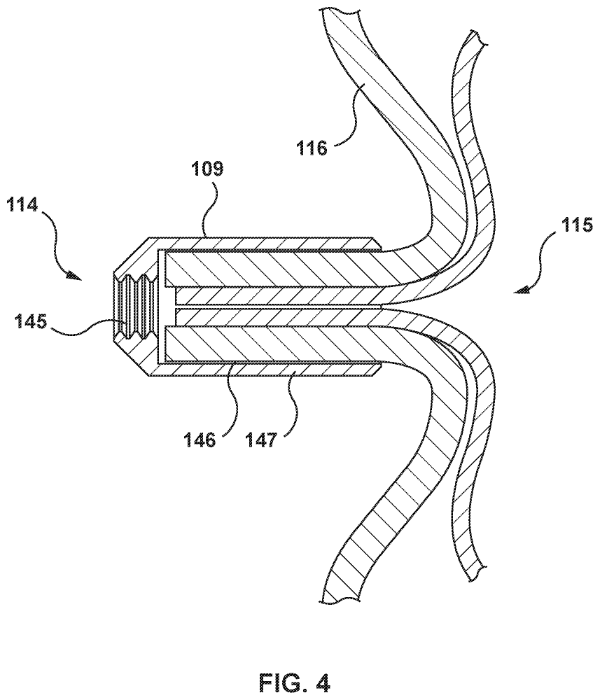

In embodiments having spokes 116 a coupler 109 may also function to contain the terminals of the spokes. FIG. 4 shows a coupler 109 that is a rigid tube having a female threaded section 145 on its proximal end 114 for mating with a male threaded coupler on a delivery tool 108 (FIG. 2B). A lumen 146 defined by the walls of the rigid tube 147 hold ends of the spokes 116, which may be for example terminals of wires used to braid the structural frame 101 (FIG. 2A) or alternative spoke elements. Spokes 116 may be firmly connected to the tube 147, for example via crimping, welding or adhesive.

Optionally, a coupler may be laser cut from a Nitinol hypotube, which may also form spokes and radially protruding retention barbs.

Covering/Seal

Lobar valves disclosed herein may further have at least one membrane (102 in FIG. 2A) connected to the structural frame 101 that functions to create an air seal of the lobar bronchus permitting air to flow only or at least predominantly through a one-way valve 103. The material of the sealing membrane 102 may further function to resist tissue ingrowth so the lobar valve may be safely removed after a prolonged period of remaining implanted. The material may be made from a material or have a layer that avoids it from sticking to itself, which facilitates transformation of the lobar valve from a collapsed delivery state to an expanded deployed state.

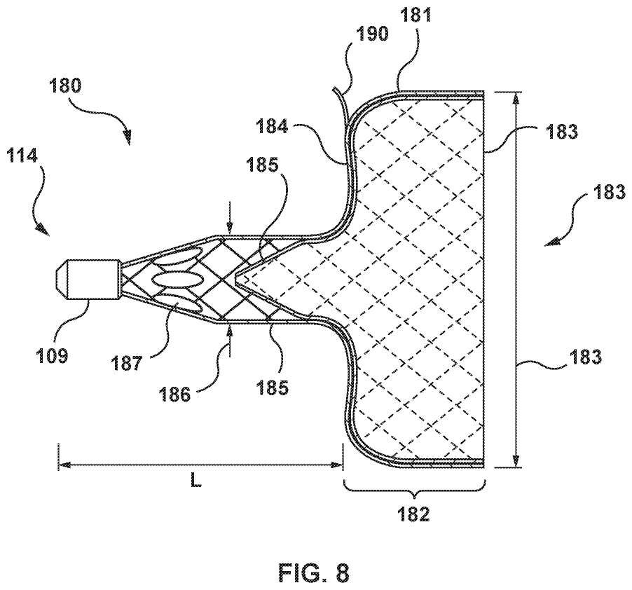

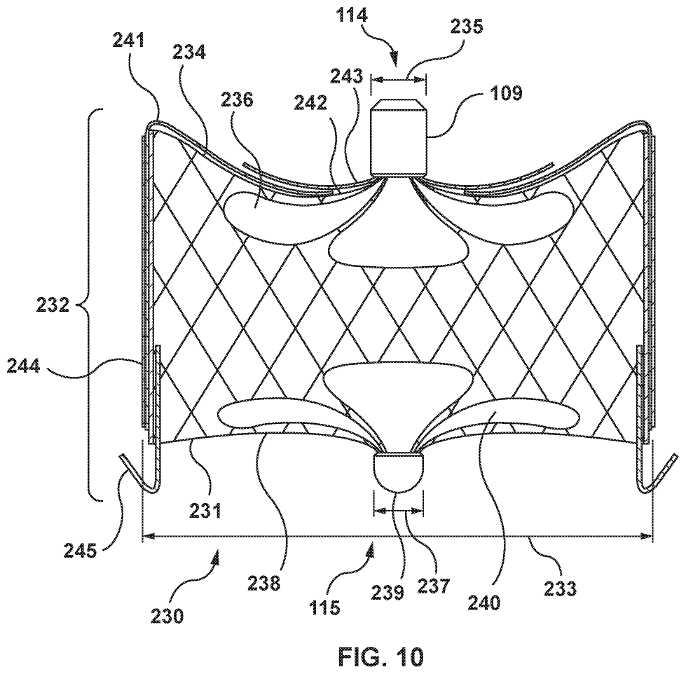

The membrane connected to the structural frame may be made from a thin, flexible, durable, foldable, optionally elastic material such as urethane, polyurethane, ePTFE, silicone, Parylene, Elast-eon™ or a blend of multiple materials. The membrane may be made by insert molding, dip coating or spray coating a mold or other manufacturing methods know in the art of medical balloon or membrane manufacture. It may be bonded to the frame for example by coating the frame, laminating over the frame, dip coating, spray coating, heat staking, bonding with adhesive, solvent bonding, or sewing. Referring to FIG. 2A as an example, a membrane 102 may cover the wall contact region 110 of the structural frame 101 and at least a portion of a luminal covering region 119 to disallow air from flow through a lumen of the bronchi except for through the one-way valve 103 and impede air from leaking around the edges between the wall contact region and an airway wall. A luminal covering region 119 may be on the distal side 115 of the wall contact region 110, or in some embodiments on a proximal side 114 (e.g., FIGS. 8 and 10). The luminal covering region 119 may be flat or have a convex shape that bulges out distal to the airway contact region 110 as shown in FIG. 2A. The extra material of a bulging luminal covering region 119 may allow the structural frame to conform to a non-circular airway cross section. The membrane material may also be partially stretchy to allow it to conform to irregular airway geometry. For example, the membrane material may be rated for up to 500% elongation.

The sealing membrane may be positioned and bonded outside the structural frame. Alternatively, a sealing membrane may have an inner membrane layer bonded to the inner surface of the structural frame as well as an outer membrane layer bonded to an outer surface of the structural frame wherein the inner and outer layers may be bonded to one another between braid wires or spokes 116 thus encapsulating at least a portion of the structural frame.

Airflow 120 as shown in FIG. 2C flows from the lobe distal to the device 100, through a valve 103, and out of the lung. The sealing membrane 102 in combination with the one-way valve 103 impedes air from flowing the opposite direction into the lobe. Optionally, the membrane may also form the one-way valve, or alternatively a one-way valve may be a separate structure connected to a structural frame or sealing membrane.

Portions of the sealing membrane 102 framed by wires of the structural frame in the airway contact region 110 may be flexible and have slack that functions to facilitate air sealing by billowing out and applying contact pressure with the airway wall over a surface area defined by the sealing membrane portions when air is passing through the device or a pressure difference is higher within the device.

The sealing membrane 102 and structural frame 101, in particular the wall contact region 110, form a contact surface area that is continuous around a circumference of a targeted airway.

In an alternative embodiment of a sealing membrane the membrane may have channels or openings that intentionally allow air to pass the seal in either direction initially after the device is implanted and gradually close to block air passage except for through a valve. For example, the channels may be positioned on the seal surface next to the airway wall and over time (e.g., a few weeks) become plugged with mucus that naturally exists in the airway. Gradual or delayed sealing could delay the evacuation of trapped air and subsequent lobar volume reduction so that shifting of the lobes of the treated lung occurs more gradually, which may be less likely to have adverse events such as pneumothorax or injury to healthy lung tissue.

Optionally, a membrane may deliver a chemical agent released slowly over time. For example, the membrane may deliver an antiseptic, antimicrobial or other agent, which may reduce the risk of infection, pneumonia, rejection or other complication. For example, a membrane may be impregnated with an agent such as an antifungal, antibacterial, antimitotic, or anti-inflammatory agent that may improve patient response to implanting the device.

Optionally, a membrane 102 may have a micropatterned surface that provides a non-stick or hydrophobic feature on the interior side (i.e., facing inward toward the axis 118) of the airway contact region 110, on the luminal covering section 119, on the valve, or a combination. The non-stick micropatterned surface may have a lubricious texture pattern which may reduce friction and repel or allow fluids such as mucous to slide off the membrane. The hydrophobic feature of the micropatterned surface may be created by nanostructures molded on to the polymeric membrane 102.

Optionally, a hydrophobic coating may be added to the interior side of the airway contact region 110, on the luminal covering section 119, on the valve, or a combination.

Optionally, a membrane 102 may have a micropatterned surface that provides increased hydrophilic character, friction or surface tension on the exterior side of the airway contact region 110.

One-Way Valve

The lobar valve 100 is adapted to provide a seal that does not allow air to flow, or at least substantially increases resistance to airflow through the targeted airway except for through the one-way valve 103. The sealing function is achieved by the membrane 102 connected to the structural frame and the sealing membrane 102 may also form the one-way valve 103. Alternatively, a one-way valve may be a separate structure bonded to the sealing membrane or structural frame. Generally, the one-way valve is adapted to allow air to flow at least predominantly in one direction, from the affected lobe and not into it.

Optionally, a valve material may be impregnated with an agent such as an antifungal, antibacterial, antimitotic, or anti-inflammatory agent that may improve patient response to implanting the device.

As an example, a one-way valve 103 may be made from a flexible, non-stick material such as an elastomeric material, urethane, polyurethane, ePTFE, silicone, Parylene or a blend of multiple materials. The one-way valve 103 may be a duckbill or Heimlich valve having a somewhat funnel shape that transitions from a distal flared end to a proximal closing end. The distal flared end may be tubular having an outer diameter that connects with the luminal covering region 119 of the sealing membrane 102. The distal flared end may have a diameter 121 in a range of 1 mm to 4 mm (e.g., 2 mm to 3 mm). The length 122 of the one-way valve 103 may be in a range of 3 to 8 mm (e.g., 5 mm). The Heimlich valve 103 includes a pair of opposed, inclined walls having ends that meet at lips at the proximal end. The lips meet at two opposed corners and may be pinched flat. The walls can move with respect to one another so as to separate at the lips and form an opening through which fluid can travel. When exposed to fluid flow in a direction represented by the arrow 120 in FIG. 2C at a cracking pressure, the walls separate from one another to form the opening through which the fluid may flow. When exposed to fluid flow in an opposite direction the lips remain closed and prevent fluid from flowing through the duckbill valve. Alternatively, other forms of one-way valves known in the art of medical devices may be used. Optionally, the lips may be normally opened at least a small amount when there is no pressure differential across the valve, which may reduce or eliminate the cracking pressure and reduce an opening response time.

Optionally, a one-way valve 103 may be adapted to provide a desired exiting air flow resistance. It may be desired to release air from the target lobe slowly to reduce a risk of pneumothorax that can be caused by rapid deflation of the lobe. Exiting air flow resistance may be inversely proportional to the valve's lumen diameter proportional to its length and may be a function of material stiffness.

Any of the lobar valve embodiments disclosed herein may optionally have a temporary reverse flow component that initially and temporary allows some air to flow from the proximal end 114 to the distal end. This feature may function to slow down the volume reduction of the targeted lobe to reduce a risk of pneumothorax associated with rapid deflation. For example, the feature may be a biodegradable or dissolvable component that hold the one-way valve 103 partially open or provide a gap between the airway contact region of a device 100 and the targeted airway wall. The component may shrink or dissolve over an initial duration of time (e.g., in a range of 3 days to 3 weeks).

Retention Mechanism

A lobar valve may have a retention mechanism such as radial contact force, radially extending barbs, a micropatterned surface on the membrane, placement distal to cartilaginous rings, radial interference, or a combination of these. The retention mechanism functions to keep the device situated and oriented in the targeted position of the patient's airway. The device may be removed by applying force (e.g., pulling, torqueing) to the coupling element or structural frame to overcome the retention force. Alternatively, the retention mechanism may be released from the airway by collapsing the lobar valve.

Radial contact force applied by the airway contact region 110 to the airway wall can help to retain the device 100 in the desired implant location in a lobar bronchus by contributing to friction. Furthermore, radial contact force may distend the airway wall creating a niche for the device to sit in. Radial contact force may be created by the elastic properties of the structural frame 101 returning to its shape set configuration, which may be larger (e.g., 5 to 20% larger) than the airway. Additional radial contact force may be created by optional spokes 116.

FIG. 2A shows radially extending barbs 104 extending from the proximal end 114 of the airway contact region 110. Alternatively, the barbs may extend from the distal end 115 or along the airway contact region 110. The barbs may be made from thin wires connected to the structural frame adapted to protrude beyond the diameter 111″ of the airway contact region 110 when the device 100 is expanded in situ. For example, the barbs may protrude up to 3 mm (e.g., about 1 mm) and be made from wire having a diameter in a range of 0.003″ to 0.008″ (e.g., about 0.005″). The wire may be superelastic Nitinol (e.g., having a transition temperature below room temperature or below body temperaturer. Alternatively, barbs may be made from the wires braided to form the structural frame 101. For example, some of the wire terminals may be shape set to become barbs or some of the closed loop ends 113 may be cut and shape set to form barbs. Alternatively, barbs 104 may be made from a laser cut tube, which may also form spokes 116.

A micropatterned surface on the polymeric membrane 102 at least in the airway contact region 110 may help to retain the device in place by resisting sliding on wet surfaces such as airway walls but not on dry surfaces such as through a delivery sheath. For example, a micropattern may be molded to the membrane using techniques known in the art (e.g., U.S. Pat. No. 8,720,047 assigned to Hoowaki, LLC). The micropatterned surface may increase water tension when contacting a wet surface which can greatly increase retention ability. The micropattern may have a plurality of pillars having height and width dimensions less than 1000 nanometers.

Placement of the device just distal to a cartilage ring in an airway may contribute to retention of the device. Cartilage rings exist in lobar bronchi in particular at the proximal end of lobar bronchi and may protrude from the airway surface where cartilage rings are absent. Since the structural frame is shape set to a larger size than the airway it elastically expands against the airway wall. To overcome the cartilage ring, the structural frame would have to reduce in size which goes against its elastically expanding nature.

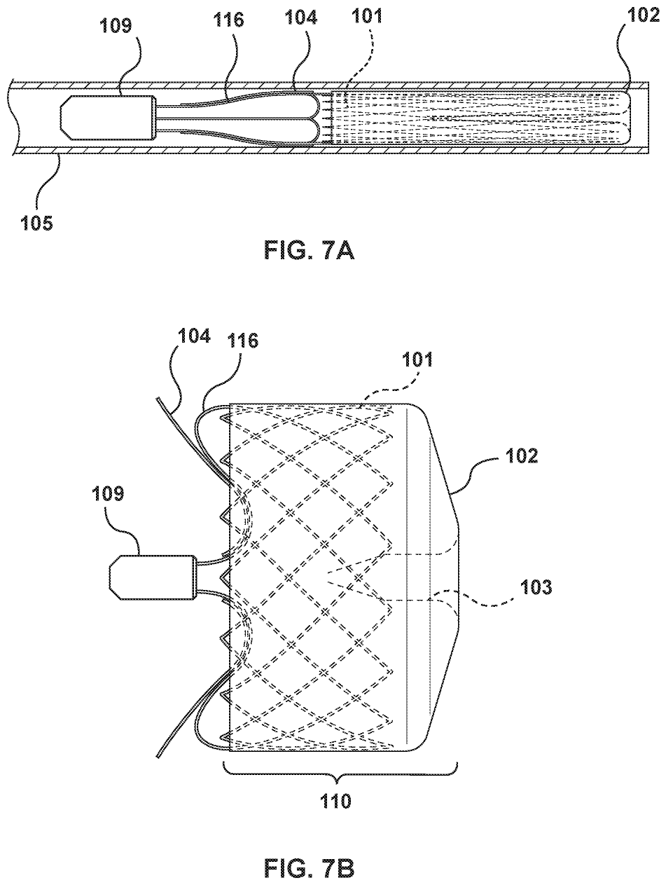

As shown in FIG. 7A in a constrained delivery state the barbs 104 may be retracted and flush with the spokes 116 and braided airway contact region 110, allowing the device to be advanced through or from a delivery sheath 105. When the lobar valve expands to in deployed state (FIG. 7B) the barbs 104 deploy to radially protrude from the airway contact region 110. The wires forming the barbs 104 may be connected to the spokes 116, for example, woven or bonded to the spokes. Alternatively, the wires forming the barbs 104 may be connected into the coupler 109, or a combination of connection to a coupler and connection to the spokes.

Alternatively, barbs 104 may be made from a laser cut hypotube. For example, a coupler, spokes and barbs may be made from a laser cut hypotube, wherein the spokes are connected to a braided structural frame forming an airway contact region.

Regardless of the retention mechanism embodied, a lobar valve 100 may be implanted and before removing the delivery tool and bronchoscope, a pull force test may be applied to the device to ensure it has been sufficiently anchored in place. With the delivery tool connected to a grasping mechanism of an implanted lobar valve, the pull force may be conducted by applying a gentle pull force on the delivery tool. A force gauge may indicate the amount of force applied to the lobar valve. If the valve becomes dislodged below a predetermined force, the retention mechanism of the stent may not suit the current implantation, a different sized device may be required, or the device may need to be repositioned.

### Example Embodiment 1 Braided Frame with Spokes

A first embodiment of a lobar valve as shown in FIGS. 2A, 2B, 5A, 5B, 6A, 6B, 7A and 7B has a braided structural frame 101 with an airway contact region 110 and radial spokes 116 connecting the airway contact region 110 to a coupler 109. A sealing membrane 102 is connected to the airway contact region 110 and extends past the distal end 115 of the airway contact region where it forms a luminal covering region 119 extending from diameter 111″ to diameter 121 and further forms a one-way valve 103. General features of these elements that are disclosed herein may apply to this embodiment.

The wires of the braided structural frame 101 have closed loop ends 113 on the distal side 115 and on the proximal side 114 the wires are gathered and shape set to form the spokes 116. The terminals of the wires are held (e.g., crimped, welded) in the coupler 109.

Barbs 104 radially protrude from the proximal end of the airway contact region 110.

Relative to the airway contact region 110, the spokes 116 may be angled proximally 114 as shown in FIG. 2A or alternatively may be perpendicular to the airway contact region 110 or angled distally as shown in FIG. 5A. Lobar valves with spokes that are angled distally position the coupler 109 at least partially in the lumen of the airway contact region 110 effectively reducing the device length 112″, which may be advantageous especially where less room in the airway is available. Inverted, or distally angled spokes may further facilitate retention.

Optionally spokes 116 may be “S” shaped spokes 155 as shown in FIG. 5B.

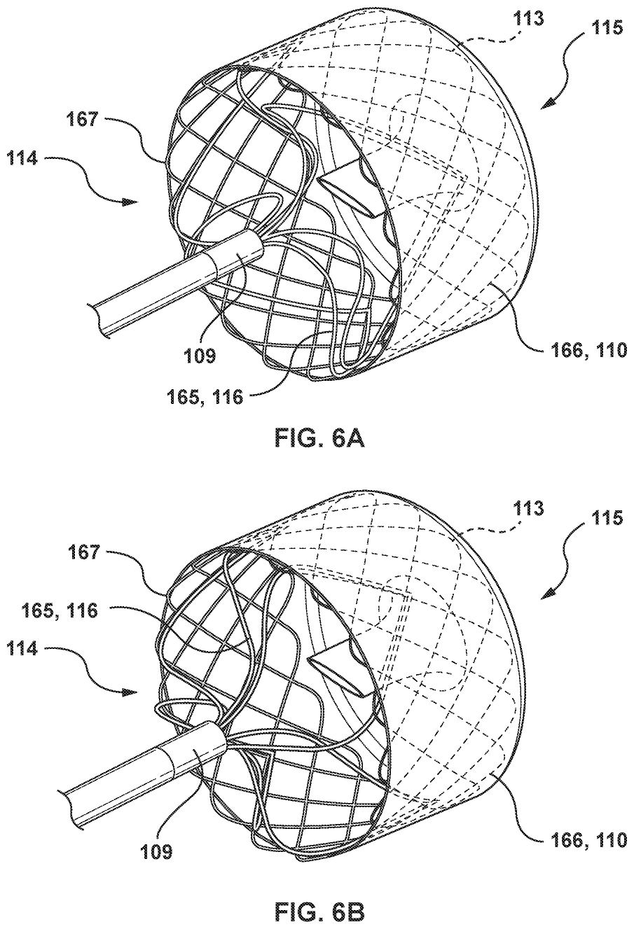

Alternatively, as shown in FIGS. 6A and 6B spokes 116 may be separate wires (e.g., Nitinol) 165 than the wire(s) forming the airway contact region 110, 166 of the structural frame. A tubular wire-braided airway contact region 110, 166 may be made with a single wire with the terminals woven into the airway contact region 166 so that both distal 115 and proximal 114 ends have closed loop ends 113, 167. For example, a lobar valve may have separate wire spokes 165 including three wires (FIG. 6A) or four wires (FIG. 6B). The wires forming the spokes 165 may be looped through a part (e.g., proximal closed loop ends 167, or other part) of the braided airway contact region 166 with both terminals connected to the coupler 109.

Alternatively, spokes and optionally a coupler or radial barbs may be made from a laser cut hypotube (e.g., Nitinol).

The optional barbs 104 may be formed from a variety of options disclosed herein such as separate wires from the braided structural frame connected to the spokes or airway contact region, wires forming the braided structural frame cut and shape set to protrude forming the barbs, or portions of the braided structural frame shape set to protrude outward.

### Example Embodiment 2 Braided Frame with Tapered Proximal End

An alternative embodiment 180 of a lobar valve is shown in FIG. 8, wherein a structural frame 181 is made from a braided wire Nitinol tube that is shape set to form an airway contact region 182 having a first diameter 183, a valve housing region 185 having a narrower second diameter 186, and a luminal covering region 184 spanning from the first diameter 183 to the second diameter 186. The proximal end of the braided tube may be connected to a coupler 109 at the proximal end of the device 114. Enlarged holes 187 may be shape set into the braided tube 181 proximal to the valve housing region 185, which may facilitate flow of air or fluids or reduce the risk of clogging the braided frame. The luminal covering region 184 of the braided frame may have an “S” shaped profile as shown, which may facilitate deployment, retraction, and radial retention force. The membrane 188 may be connected to the airway contact region 182 of the braided frame, be open at the distal end 115 of the device 180, be connected to or at least span the luminal covering region 184, and form a one-way valve 189 that is contained in the valve housing region 185. Radially protruding barbs 190 optionally may be connected to the structural frame and may be a variety of barb embodiments or positions disclosed herein.

### Example Embodiment 3 Braided Frame Open on Both Ends

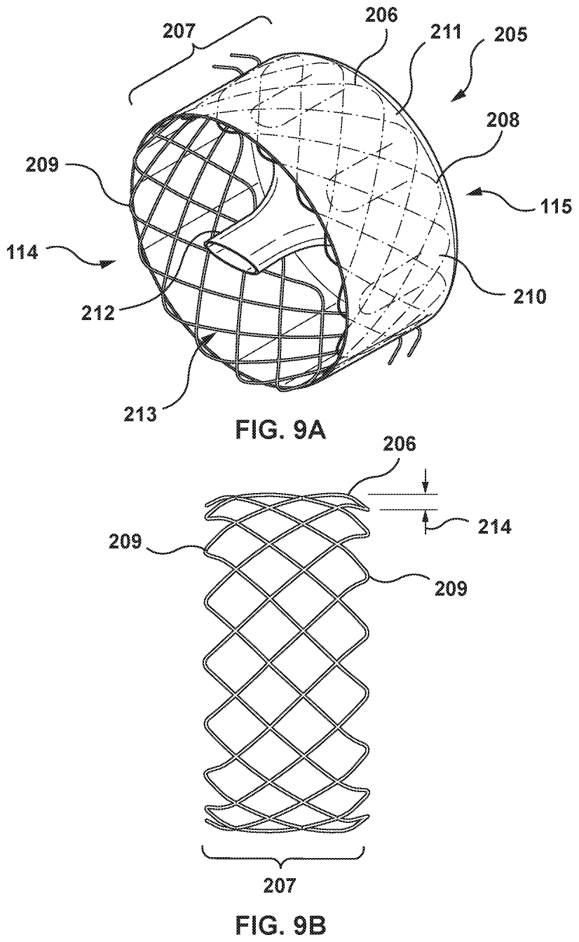

Another alternative embodiment of a lobar valve 205 is shown in FIG. 9A, wherein a braided structural frame 206 forms a tubular airway contact region 207 with an open distal end 115 and open proximal end 114. The distal end of the braid 115 and the proximal end of the braid 114 both may have closed loop ends 208 and 209. The braid 206 may be made from one wire with both terminals woven into the airway contact region 207. Optional radially protruding barbs 210 may be connected to the braided structural frame 206. For example, a pair of barbs 210 may be made from a Nitinol wire that is woven into the braid 206 with terminals shape set to radially protrude as shown. A membrane 211 may be connected (e.g., solvent bonded, dip coated, glued, sewn) to the airway contact region 207 of the braided structural frame 206, span a luminal covering region, and form a one-way valve 212 held in a lumen 213 defined by the airway contact region 207 of the structural frame 206.

Optionally, as shown in FIG. 9B the distal and proximal closed loop ends 208 and 209 of the braided structural frame 206 may be bent inward a distance 214 (e.g., 0.25 mm to 1 mm) as shown which may reduce a risk of irritating the tissue of the airway wall by reducing friction applied to the tissue by the closed loop ends during movement.

### Example Embodiment 4 Braided Frame Closed on Both Ends

Another alternative embodiment of a lobar valve shown in FIG. 10 has wire braided structural frame 231 having a tubular airway contact region 232 with a first diameter 233 adapted for placement in a lobar bronchus. At the proximal end 114 of the device 230 the wires of the braided structural frame 231 are shape set to span the luminal covering region 234 from the first diameter 233 to a narrower second diameter 235 where the wires are connected to (e.g., crimped into or welded to) a coupler 109. The wire braid in the luminal covering region 234 may have shape set holes 236 that may facilitate passage of air or other fluids. Similarly, at the distal side 115 of the device 230 the wires of the braided structural frame 231 span a luminal covering region 238 from the first diameter 233 to a narrower third diameter 237 where the wires may be crimped together in a distal crimp 239. The distal luminal covering region 238 optionally may also have shape set holes 240 to facilitate passage of air or other fluids. The proximal and distal luminal covering regions 234, 238 with shape set holes 242, 240 are an alternative form of spokes that connect the airway contact region to a hub (e.g. coupler 109 or crimp 239. Alternatively, spokes may be configured in other various embodiments of spokes disclosed herein which may be both on the proximal 114 and distal ends 115.

A sealing membrane 241 may be connected to the braided structural frame at least partially over the airway contact region 232 and a portion of the proximal luminal covering region 234 leaving an uncovered part 242 of the luminal covering region 234. A separate membrane flap 243 connected to the coupler 109 or structural frame temporarily covers the gap 242 and overlaps a portion of the membrane 241 when air pressure is higher on the proximal end 114 than the distal end 115. The flap 243 opens when pressure is higher on the distal end 115 than the proximal end 114. Thus, the flap 243 and membrane 241 act as a one-way valve.

Alternatively, the membrane 241 may partially cover the distal luminal covering region 238 and a one-way valve may be formed with a flap at the distal end also adapted to preferentially allow air flow from the distal to proximal ends (not shown).

Optionally, the membrane 241 at least on the exterior portion of the airway contact region 232 may have a molded micropattern 244 to increase retention in the airway.

Optionally, radially protruding barbs 245 may be connected to the braided structural frame 241. The barbs 245 may be one or more of the various embodiments of radially protruding barbs disclosed herein.

The braided structural frame forming both proximal 234 and distal 238 luminal covering regions may have increased strength or radial contact force with the airway wall in situ.

### Example Embodiment 5 Braided Frame Forming an Inner and Outer Tube

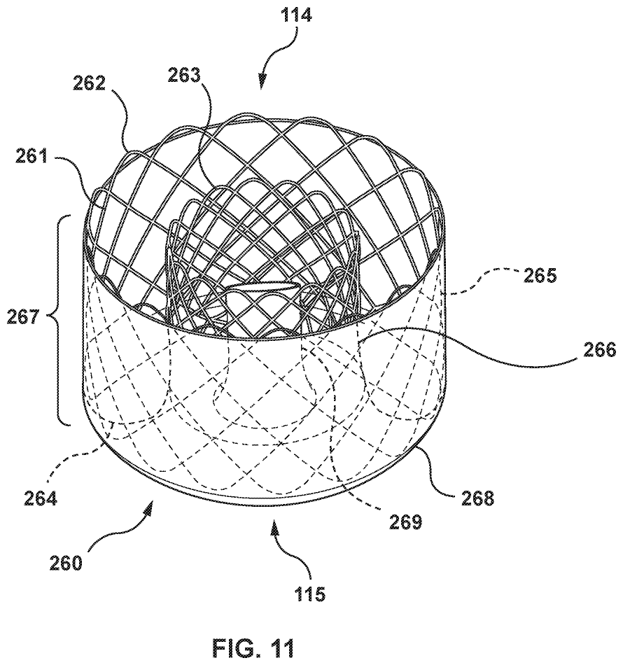

Another alternative embodiment of a lobar valve 260 is shown in FIG. 11 wherein a tubular braided structural frame 261 is a tube with a first end 262 and a second end 263 that is folded in on itself forming an outer tubular region 265 and an inner tubular region 266 spanned by a luminal covering region 264 on the distal end 115 of the device 260. Alternatively, a luminal covering region may be on a proximal end 114. The outer tubular region 265 forms an airway contact region 267. Optionally the tubular braided frame 261 may be made from one Nitinol wire braided into a tube that is shape set to form the outer and inner tubular regions 265, 266 and the wire terminals may be woven into the tube so they are not exposed. Optionally both the first end 262 and second end 263 may have closed loop ends. Optionally, at least some of the closed loop ends on the first end 262 may be bent outward to function as radially protruding retention barbs. Alternatively or optionally, radially protruding retention barbs may be made by cutting some of the closed loop ends on the outer first end 262, or made by connecting separate wires to the structural frame.

A sealing membrane 268 may be connected to the braided structural frame 261 on at least a portion of the airway contact region 267, where the membrane may optionally have a micropatterned surface on the exterior of the membrane to enhance retention in an airway. The membrane may also cover the luminal covering region 264 and form a one-way valve 269 (e.g., Heimlich or duckbill valve) in a lumen defined by the inner tube 266.

The embodiment shown in FIG. 11 may not have a separate component for a coupler but instead the second end 263 may connect to a delivery tool for delivery, deployment, or retraction of the device 260.

Delivery Tool

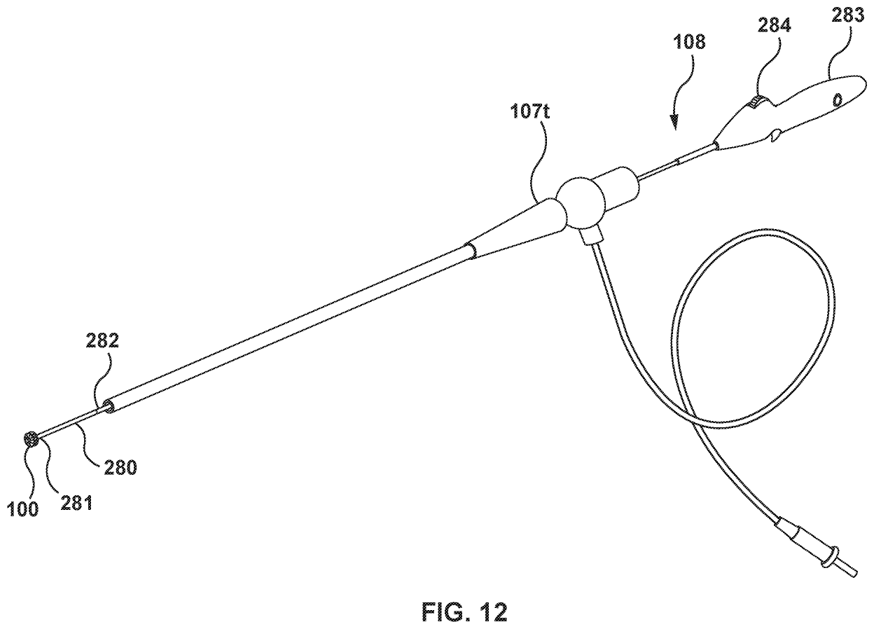

As shown in FIG. 12 a delivery tool 108 for delivering a lobar valve (e.g., 100) through a working channel of a bronchoscope 107 may have a delivery shaft 280, which may be a flexible, elongate, tubular or rod structure, with a coupling element 281 at its distal end that is shaped to couple with the coupler (e.g., 109) of the lobar valve, a delivery sheath 282, and a handle 283 at its proximal region. For example, the coupling element 281 may be a male threaded rod adapted to mate with a female threaded opening 145 of a lobar valve coupler 109 (FIG. 4). A delivery shaft may be flexible to bend and navigate through a bent bronchoscope in a tortuous airway yet be longitudinally and circumferentially non-compliant to resist stretching, compression or kinking so it transmits motion from the proximal end (e.g., handle) to the coupling element 281 and to the lobar valve 100. A delivery shaft may be made from a polymer and have an embedded laser cut tube or tight wire coil.

An alternative embodiment of a delivery shaft, may have a central lumen, which may be used for delivery over a guidewire or to pass over or deliver other instruments such as an endoscope. Optionally a delivery shaft may have a mandrel extending distally, which may be used to hold a valve to the delivery shaft, to add coupling force, to target a coupler of a lobar valve when retrieving it or to adjust its position.

Optionally, the delivery tool may have a delivery sheath 282 used in conjunction with the delivery shaft 280. The sheath may constrain the valve in a delivery state during delivery through a working channel as shown in FIG. 2B. A distal section (e.g., about 10 cm of the distal end) of the delivery sheath may be relatively more flexible allowing it to bend and traverse a bronchoscope that is bent at its distal end to navigate a tortuous airway. The delivery sheath may be non-compliant over its full length to resist compression or stretching. The delivery sheath may be circumferentially non-compliant at least at its distal end so it can contain and constrain a lobar valve in its contracted delivery state. A laser cut steel tube may be embedded in a polymer such as Pebax at its distal section to provide hoop strength and circumferential non-compliance. The delivery sheath may be made of a polymer such as Pebax or polyimide with an embedded wire braid or wire coil to resist compression, stretching or kinking. The delivery sheath 282 may have an outer diameter sized to slidably pass through the bronchoscope working channel 106 (e.g., to fit a 2.8 mm the sheath may have an outer diameter in a range of 2.0 mm to 2.7 mm). The sheath may have an inner diameter in a range of 1.5 mm to 2.5 mm.

Optionally, the delivery tool may have a handle 283 at a proximal region that has an actuator (e.g., thumb lever) that controls a sliding translational movement of the shaft 280 with respect to the sheath 282 facilitating one-handed control for advancing a valve out of a sheath or retracting it into the sheath. For example, a sheath 282 may be connected to the handle body and a shaft 280 may be slidably engaged in the sheath and connected to a gear that is movable (e.g., rotation or translation) within the handle and moved by a mating gear connected to an actuator such as a thumb lever, slider, or rotary dial. The handle may have one or more actuators that move the delivery shaft and control the position of the lobar valve from a fully contained position as shown in FIG. 2B to a partially deployed position with the couplers connected to a fully deployed and released position as shown in FIG. 2C. A first actuator 284 may be used to pull the device 100 into the delivery sheath (FIG. 2B), optionally with help of a loading tool. The first actuator may be used to advance and retract the sheath 282 relative to the shaft 280 to partially deploy the lobar valve 100. This step may be used to assess position and fit within a target airway while visualizing deployment through a lens of the bronchoscope 107. The first actuator 284 may stop at the position of stage 1 before fully releasing the device. A second actuator 285 such as a trigger may be used to fully release the lobar valve 100, for example by rotating the shaft 280 to unscrew the delivery tool coupler 281 from the device coupler 109. The first and second actuators may be ergonomically oriented on the handle 183 to be used with one hand for example the first actuator may be oriented for use with a thumb and the second actuator may be oriented for use with an index finger of the same hand.

Kit

Optionally a lobar valve may be provided preloaded in a delivery sheath, optionally disposable, in its constrained delivery state and coupled with a delivery shaft as shown in FIG. 2B. Alternatively, a lobar valve may be provided coupled to a delivery tool with the lobar valve advanced out of a delivery sheath in its unconstrained state for example as shown in FIG. 2A. The assembly may be provided contained in a sterilized package with instructions for use. A lobar valve provided partial deployed may facilitate visual inspection and avoid material deformation caused by prolonged constraint. Optionally, the unconstrained lobar valve may be held in a loading tool, for example having a funnel, that facilitates contraction of the device into a contracted delivery state in a delivery sheath.

Delivery

A method of use may involve the following delivery steps:

From a CT scan, measurements are taken to confirm intended valve placement location, target airway diameter and length;

An appropriately sized lobar valve is chosen to match the measured airway size.

The lobar valve is visually inspected prior to loading into a delivery sheath;

A bronchoscope is advanced through the patient's endotracheal tube to the targeted lobar airway;

The lobar valve in the delivery sheath is advanced distally through a working channel of the bronchoscope;

The distal end of the delivery system is advanced distally out of the working channel to a desired valve position in the target airway;

While holding the bronchoscope in position relative to the airway the delivery sheath is retracted proximally relative to the shaft and lobar valve to deploy the lobar valve to its expanded but coupled position;

The position, fit, alignment, and seal may be visually inspected through the lens of the bronchoscope. The delivery system may be pulled gently to confirm mechanical anchoring or engagement of the valve against the airway wall;

If position, fit, alignment, seal and anchoring are not satisfactory optionally push or pull the delivery system to adjust;

If position, fit, alignment, seal and anchoring are still not satisfactory retract the lobar valve at least partially back into the delivery sheath;

The delivery sheath and lobar valve may be repositioned and redeployed;

If the position, fit, alignment, seal and anchoring are satisfactory the lobar valve may be disengaged from the coupler of the delivery system;

The delivery system may be removed from the patient;

The lobar valve may be visually inspected through the lens of the bronchoscope;

The bronchoscope may be removed from the patient.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A flow control device for a lung of a living patient, the flow control device comprising
a support frame, while in an expanded configuration, including:
an outer cylindrical section with a distal circumferential edge, wherein the outer cylindrical section is sized and configured to contact a lobar bronchus in a lung of a patient;
an inner cylindrical section with a distal circumferential edge, and
an annular section spanning and connecting proximal ends of the outer and inner cylindrical sections,
a sealing membrane covering and/or attached to the distal circumferential edge of the outer cylindrical section and configured to span a cross sectional area of the lobar bronchus, and
a one-way valve attached to or integrated with the sealing membrane, wherein the one-way valve includes an inlet configured to be oriented towards a downstream flow region of the lobar bronchus, wherein a flow direction is of air inhaled into the lung and flowing through the lobar bronchus, and an outlet configured to be oriented towards an upstream flow region of the lobar bronchus, and wherein the one-way valve extends into the inner cylindrical section.

2. The flow control device of claim 1 wherein the sealing membrane covers at least a portion of an outer surface of the outer cylindrical section.

3. A flow control device for a bronchial passageway comprising:
a proximal end, a distal end, a central axis;
a structural frame, wherein the structural frame is transitionable between a compressed configuration and an unconstrained expanded configuration;
a sealing membrane connected to the structural frame; and
a one-way valve,
wherein the structural frame comprises at least one of a wire braid, mesh or woven material arranged in a tubular shape.

4-5. (canceled)

6. The flow control device of claim 3, wherein at least one wire forming the wire braid, mesh or woven material has a diameter in a range of 0.003″ to 0.008″.

7. (canceled)

8. The flow control device of claim 3, wherein the wire structural frame comprises a braid angle in a range of 35° to 55° in the unconstrained expanded configuration.

9.-15. (canceled)

16. The flow control device of claim 3, wherein the at least one wire of the structural frame forms closed loop ends at the distal end of the flow control device.

17. The flow control device of claim 16, wherein the closed loop ends have a smaller angle than a braid angle of the at least one wire.

18. (canceled)

19. The flow control device of claim 17, wherein the closed loop ends comprise short and long closed loop ends arranged in an alternating pattern.

20.-22. (canceled)

23. The flow control device of claim 16, wherein the at least one wire comprises wires braided or woven into the wire structural frame forming closed loop ends on the distal end.

24. The flow control device of claim 16, wherein the at least one wires has terminals that are gathered to form spokes at the proximal end of the support structure.

25.-28. (canceled)

29. The flow control device of claim 3, further comprising spokes and a coupler, wherein the spokes connect the coupler to the structural frame.

30. The flow control device of claim 29, wherein the spokes extend from the coupler at an angle to a central axis of the wire support structure in a range of 0° to 40° in the expanded configuration.

31.-32. (canceled)

33. The flow control device of claim 29, wherein the spokes comprise an S-shaped curve.

34. The flow control device of claim 29, wherein the spokes are longer than the radius of the flow control device in its unconstrained expanded configuration.

35. The flow control device of claim 29, wherein the spokes are inverted into a lumen of the structural frame when in the unconstrained expanded state.

36. The flow control device of claim 29, wherein the spokes are invertible into a lumen of the structural frame when in the unconstrained expanded state.

37.-42. (canceled)

43. The flow control device of claim 3, wherein the sealing membrane is configured to direct a majority of bronchial passageway airflow through the one-way valve.

44. (canceled)

45. The flow control device of claim 3, wherein the sealing membrane comprises an airway wall contact region, and a luminal covering region.

46.-48. (canceled)

49. The flow control device of claim 3, wherein the sealing membrane occludes a part of an opening of the structural frame while the one-way valve is closed.

50.-52. (canceled)

53. The flow control device of claim 49, wherein a portion of the sealing membrane that occludes the opening has an area larger than the cross section of the opening.

54. (canceled)

55. The flow control device of claim 3, wherein the sealing membrane comprises a hydrophilic micropatterned surface.

56. The flow control device of claim 55, wherein the hydrophilic micropatterned surface is on an external surface of the sealing membrane where the sealing membrane is connected to a contact area of the support structure configured to contact the bronchial passageway.

57. (canceled)

58. The flow control device of claim 55, wherein the micropatterned surface is on at least one of an internal surface of the sealing membrane or the one-way valve.

59. The flow control device of claim 55, wherein the hydrophilic micropatterned surface comprises nanostructures molded on to the sealing membrane.

60.-66. (canceled)

67. The flow control device of claim 3, wherein the one-way valve and the sealing membrane are a single-piece component.

68. The flow control device of claim 3, wherein the one-way valve is a duckbill or Heimlich valve arranged on to allow air to flow predominantly from the distal end to proximal end.

69. The flow control device of claim 3, wherein the one-way valve is connected to a luminal covering region of the sealing membrane.

70. The flow control device of claim 3, wherein the one-way valve comprises a distal flared end having a diameter in a range of 1 mm to 4 mm.

71. The flow control device of claim 3, wherein the one-way valve comprises a length in a range of 3 mm to 8 mm.

72. The flow control device of claim 3, wherein the one-way valve comprises lips that are normally open when there is no pressure differential across the on-way valve.

73.-133. (canceled)

134. An assembly of an air flow control device and an insertion tool for a bronchial passageway comprising:
an air flow control device, wherein each of the air flow control devices includes:
a one-way valve;
a structural frame, wherein the structural frame is expandable from a collapsed configuration to an expanded configuration;
a sealing membrane mounted to at least a distal portion of the structural frame, wherein the sealing membrane forms an enclosed wall defining at least a portion of an airflow passage through the flow control device, and the one-way valve is included in the airflow passage, and
a first coupler at a proximal end of the airflow control device;
a delivery sheath configured to be positioned in a bronchial passageway, wherein the delivery sheath includes a distal end, wherein the air flow control device, while in the collapsed configuration, is within the delivery sheath;
a delivery shaft within the delivery sheath and extends through the delivery sheath towards the distal end; and
a second coupler at the distal end of the delivery shaft, wherein the second coupler is configured to securely engage the first coupler,
wherein the delivery shaft is configured to advance through the delivery sheath to push the air flow control device from the distal end of the delivery sheath and into the bronchial passageway,
wherein the air flow control device is configured to expand from the collapsed configuration into the expanded configuration after the air flow control device is pushed out of the delivery sheath, and
wherein the air flow control device is configured to automatically release from the second coupler when an actuator on a handle of the assembly is actuated.
